(19) United States
(12) Patent Application Publication
(10) Pub. No.: US 2015/0119792 A1
(43) Pub. Date: Apr. 30, 2015

(54) LIGHT DEGRADABLE DRUG DELIVERY SYSTEM FOR OCULAR THERAPY

(71) Applicant: The Regents of the University of California, Oakland, CA (US)

(72) Inventors: Adah Almutairi, La Jolla, CA (US); Viet Anh Nguyen Huu, San Diego, CA (US); Cathryn McFearin, San Diego, CA (US); Enas Mahmoud, San Diego, CA (US)

(73) Assignee: The Regents of the University of California, Oakland, CA (US)

(21) Appl. No.: 14/399,504

(22) PCT Filed: Mar. 8, 2013
(86) PCT No.: PCT/US2013/040217

§ 371 Date: Nov. 6, 2014

Related U.S. Application Data
(60) Provisional application No. 61/644,403, filed on May 8, 2012.

(51) Int. Cl. A61F 9/00; A61K 41/00; A61K 9/00; A61N 5/06; A61K 9/51

(52) U.S. Cl. CPC A61F 9/0017; A61N 5/0613; A61K 9/513; A61K 9/0048; A61K 9/0051; A61K 41/00; A61N 2005/0626; A61N 2005/067
USPC 604/21; 604/20; 604/187; 514/772.3; 424/429

(57) ABSTRACT

A system and method for delivering a payload to ocular tissue includes a solution of light-degradable nanoparticles encapsulating the payload. The solution may be introduced to the ocular tissue by way of injection or through a contact lens into which the solution is embedded. A light source delivers a beam of light to the ocular tissue at the location where the solution was introduced to initiate breakdown of the particles, releasing the payload. The light source may be a laser, LED, LCD or arc lamp emitting in the ultraviolet light range.

### RELATED APPLICATIONS

This application claims the benefit of the priority of U.S. Provisional Application No. 61/644,403, filed May 8, 2012, which is incorporated herein by reference in its entirety.

### GOVERNMENT RIGHTS

This invention was made with government support under Grant No. 1DP20D006499-01 awarded by the National Institutes of Health. The government has certain rights in the invention.

### FIELD OF THE INVENTION

The present inventions relate to a system and method for delivering a therapeutic for treatment of ocular disease and more particularly to a light-triggered ocular delivery which utilizes a polymer that degrades upon exposure to specific wavelength(s) of light.

### BACKGROUND OF THE INVENTION

The most effective treatment for a number of ocular diseases such as glaucoma, age related macular degeneration, and diabetic retinopathy requires a therapeutic to reach the posterior segment of the eye, which is usually achieved by way of ocular injection into the vitreous humor. Repeat administration using this method, such as would be necessary for controlled or sustained drug release or delivery, can result in hemorrhaging, retinal detachment, or cataracts. Others techniques for administering therapeutics include eye drops, ocular implant or injection of a therapeutic encapsulated in a non-light sensitive polymer particle or micelle.

The eye is composed of multiple tissue types, i.e., epithelium, muscle, immune cells, neural cells, and blood vessels, to name a few. Ocular diseases can affect many of these tissues at once. Nanosized carriers like micro/nano-suspensions, liposome, niosome, dendrimer, nanoparticles, ocular inserts, implants, hydrogels and prodrug approaches have been developed for controlled drug delivery to the eye. These systems offer manifold advantages over conventional systems as they increase the efficiency of drug delivery by improving the release profile and also reduce drug toxicity. Conventional delivery systems can be diluted with tears, washed away through the lacrimal gland and usually require administering at regular time intervals whereas nanocarriers release drug at constant rate for a prolonged period of time and thus enhance its absorption and site specific delivery. Ocular delivery of therapeutic nanoparticles has the potential to greatly increase the ability to maintain vision. Existing and recently-developed methods involving the use of nanotechnology have been described in the literature, including Diebold and Calonge, “Applications of nanoparticles in ophthalmology”, Prog Retin Eye Res., 2010 November; 29(6):596-609; Behar-Cohen, “Drug delivery to the posterior segment of the eye”, Med Sci (Paris) 2004 June-July; 20(6-7):701-706; Wadhwa et al., “Nanocarriers in Ocular Drug Delivery: An Update Review”, Curr Pharm Des., 2009, 15(23): 2724-2750; Lavik et al., “Novel drug delivery systems for glaucoma”, Eye, 2011, 25(5): 578-586; Patel, et al., “Ophthalmic Drug Delivery System: Challenges and Approaches”, Systematic Reviews in Pharmacy, 2010, 1(2): 113-120; Kuno and Fujii, “Recent Advances in Ocular Drug Delivery Systems”, Polymers 2011, 3(1): 193-221; and Prow, “Toxicity of nanomaterials to the eye”, Wiley Interdiscip Rev Nanomed Nanobiotechnol, 2010 July-August; 2(4): 317-33.

### BRIEF SUMMARY OF THE INVENTION

According to the present invention, a drug delivery system and method uses light directed into the subject's eye to trigger degradation of a polymer shell that releases an encapsulated ocular therapeutic payload upon exposure to specific wavelength(s). The encapsulated therapeutic may be a small molecule drug, protein, or peptide in a nano- or microparticle composed of this polymer. Administration to the eye may be achieved via a number of possible routes such as intra-vitreal injection, sub-conjunctival injection, or embedded in a contact lens applied to the eye. Controlled release of the therapeutic is achieved by selection of frequency and duration of irradiation as well as precise placement of the carriers in or on the eye.

The inventive method allows spatial and temporal control of the release of a therapeutic to the eye. Association of the therapeutic with the light-degradable polymer in a form such as a particle allows the therapeutic to remain in the eye for a longer duration without being cleared. Subsequently, using light irradiation to achieve release within the eye after the initial injection facilitates controlled and non-invasive dosing of the desired therapeutic without the possible detrimental side effects of repeated injections. Additionally, the light-degradable polymers are generally hydrophobic, which can greatly improve the delivery of some small molecule drugs that have otherwise been unsuitable for treatment due to their incompatibility with common formulations such as eye drops.

Some existing and common treatments of ocular diseases such as glaucoma are achieved through diffusion of a drug through the anterior segment of the eye. Such administration may be effectively accomplished by incorporating the light degradable polymer particles into a hydrogel contact lens. Application of the lens to the eye would then allow for triggered release to the anterior segment of the eye after irradiation.

The light degradable polymers are synthesized using previously published methods. Particles tunable through the nano- to micro-size regime may be formulated through different techniques depending on the cargo/therapeutic of choice. The particles can encapsulate a wide range of different possible therapeutics ranging from small molecule drugs to large proteins by tailoring the formulation process. Particles are then administrated via the most appropriate delivery method for the therapeutic being used. For intra-vitreous injection, a concentrated solution of particles in saline is delivered using a very small bore needle. The eye is then irradiated with a dose of light. The wavelength, power, and duration of the dose are dependent on the properties of the light degradable polymer itself. After irradiation, the polymer particle will begin to degrade into small fragments and begin releasing the therapeutic cargo inside until the particle no longer remains intact. Depending on the retention time of the particle in the eye, repeat light irradiation doses can be administered for a more sustained delivery without further need for invasive injections.

The same principles of polymer and thus particle degradation and release can be applied to other administration routes such as topical formulations, contact lens application, or sub-conjunctival injection.

In one aspect of the invention, a system is provided for delivering a payload to ocular tissue, where the system includes a solution comprising light-degradable nanoparticles encapsulating the payload; means for introducing the solution into the ocular tissue; a light source for delivering a beam of light to the ocular tissue; at least one beam adjusting optical element for controlling focus and beam size within the ocular tissue; and a system controller for providing control signals to the light source, wherein the control signals comprise selection of an emission wavelength, an emission intensity and an exposure duration, and wherein the emission wavelength is adapted to induce at least particle degradation of the nanoparticles to release the payload to the ocular tissue.

In one embodiment, the means for introducing the solution comprises a syringe and needle for intra-ocular injection, while in another embodiment the solution is introduced via a contact lens having the solution incorporated therein. The light source may be a laser, LED, LCD or arc lamp emitting in the ultraviolet light range.

In another aspect of the invention, a method is provided for delivering a payload to ocular tissue including the steps of synthesizing a particle wherein the particle further comprises a light-degradable polymer and a payload; incorporating the particle in a solution; administering the solution to the ocular tissue; and irradiating the ocular tissue comprising said particle with light having a wavelength adapted to induce degradation of the particle; wherein the particle is disrupted in situ following absorption of the light. In one embodiment, the light is ultraviolet light. The particle may be formed from a polymer having a self-immolative backbone.

In still another aspect of the invention, a drug-delivery agent for delivering a payload to ocular tissue includes light-degradable nanoparticles suspended in a solution, wherein the light-degradable nanoparticles are adapted to degrade upon exposure to light and release the payload into ocular tissue to which the nanoparticles have been introduced. In a preferred embodiment, the nanoparticles are polymers having a self-immolative backbone.

### BRIEF DESCRIPTION OF THE DRAWINGS

FIGS. 1A and 1C are diagrammatic views of the system for light-triggered delivery; FIG. 1B is a block diagram of an exemplary process for treatment of ocular diseases according to the present invention.

FIGS. 2A and 2B illustrate data from initial in vivo testing of the invention, where FIG. 2A shows measured intra-ocular pressure following injection and FIG. 2B shows electroretinography results.

FIG. 3 is a series of bright field and fluorescein channel photomicrographs of Raw 264.7 cells with FDA-containing nanoparticles with and without UV irradiation, respectively; the right column is a control.

FIG. 4 is a graph showing measured fluorescence of Raw 264.7 cells with and without UV irradiation.

FIG. 5 is a series of bright field and fluorescein channel photomicrographs of retinal cells incubated with FDA-containing nanoparticles with and without UV irradiation, respectively; the right column is a control.

FIG. 6 is a graph showing measured fluorescence of retinal cells with and without UV irradiation after injection with dye-containing light-degradable nanoparticles.

FIG. 7 is fluorescent microscope images of retinal flat mounts of non-irradiated (left) and irradiated (right) eyes after injection with dye-containing light-degradable nanoparticles.

FIG. 8 is a series of fluorescent microscope images of cryosections of the posterior ocular cup with and without UV irradiation after injection with dye-containing light-degradable nanoparticles.

FIG. 9 is microscopic images of a retinal flat mount 3 days post-injection with and without UV irradiation.

### DETAILED DESCRIPTION

As used herein, the term “particle” refers to small particles assembled according to embodiments of the present invention. The term “particle” may refer to nanoparticles or microparticles or both.

The term “microparticle”, as used herein, generally refers to a particle between 0.1 and 200 micrometers in size.

The term “nanoparticle”, as used herein, generally refers to discrete structures that are at least under 200 nm in diameter. The term “nanoparticle” may also refer to particles that are between 1 nm and 100 nm in diameter. Some of the novel properties associated with nanoparticles, which differentiate them from bulk materials, is generally associated with their size being less than 100 nm.

Microparticles and/or nanoparticles can be formed by a wide variety of means and with widely varying compositions. Examples include hydrogels such as acrylamide micelle polymerization. The may also be created from such diverse substances as poly(D,L) lactides; poly (lactic acid) (“PLA”); poly (D,L glycolide) (“PLG”); poly(lactide-co-glycolide) (“PLGA”); and poly-cyanoacrylate (“PCA”). Microparticles and/or nanoparticles can also be created from various forms of micelles/liposomes; such micelles/liposomes can be assembled via emulsions or through a process of depositions. Acrylamide hydrogels, such as ones made from N-isopropylacrylamide (NIPAAm) and acrylamide (AAm) have been made incorporating gold-gold sulfide nanoshells designed to strongly absorb near-infrared light, e.g., wavelengths between 800 and 1200 nm. When these nanoparticles are irradiated, the temperature is increased, causing in turn the release of associated molecular cargo.

The preferred light-degradable polymers used in the present invention may be synthesized using previously published methods. See e.g., international patent application PCT/US2010/04996 published as WO2011/038117 “Chemically Amplified Response Strategies for Medical Sciences”, which is incorporated herein by reference. Particles tunable through the nano- to micro-size regime may be formulated through different techniques depending on the cargo/therapeutic of choice as well as the desired sensitivity to a particular radiation source and the duration of treatment. Additional discussion of light-degradable polymers can be found in Grossman, et al., “Low Power Upconverted Continuous-Wave Near-IR Light for Efficient Polymer Degradation”,

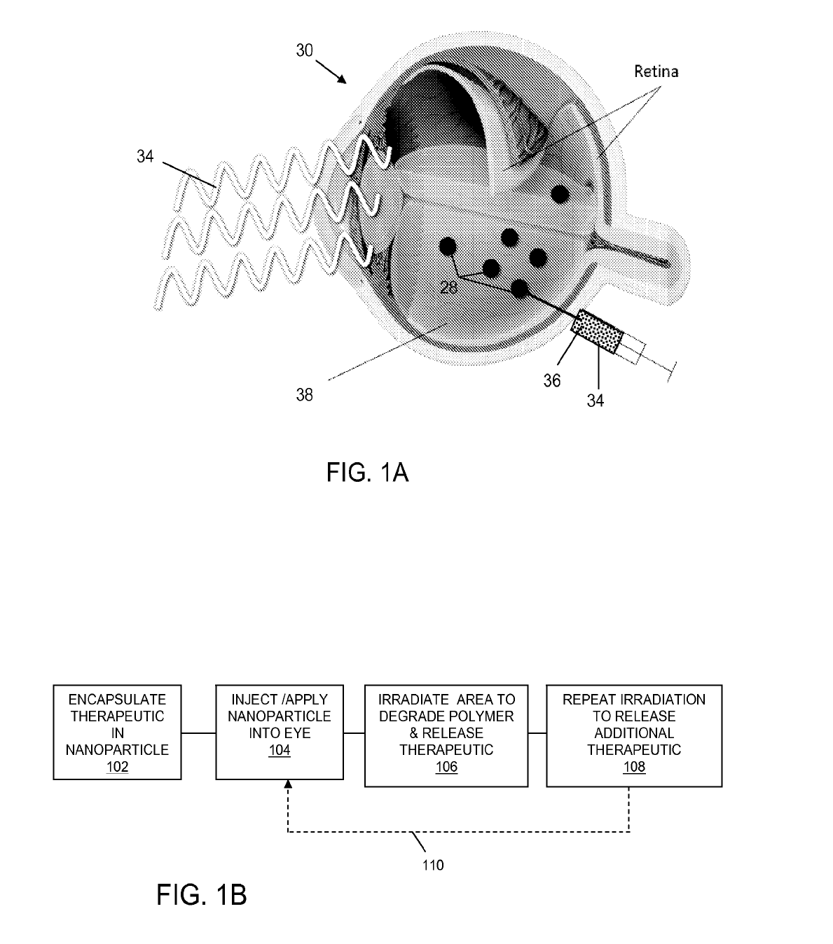

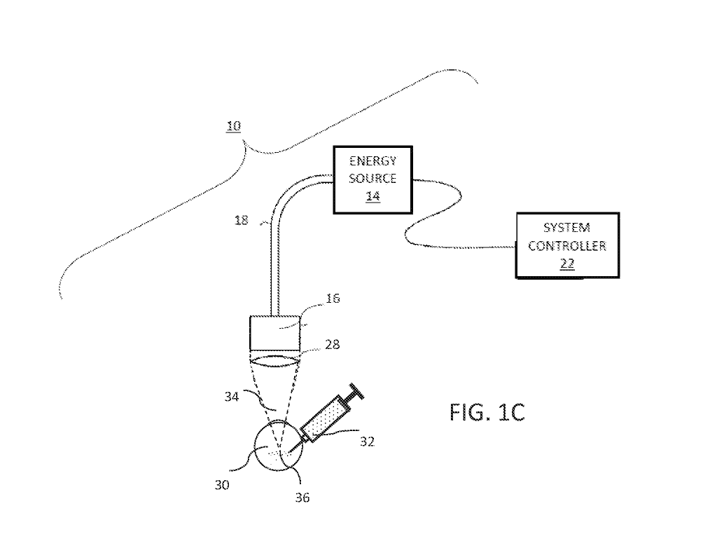

FIGS. 1A and 1B illustrate the inventive system and method for delivery of ocular therapy using light-degradable nanoparticles. In step 102, the appropriate therapeutic for the condition to be treated is encapsulated in the nanoparticles 36. The nanoparticles 36 may then be administrated via the most appropriate delivery method for the therapeutic being used. In the illustrated example, in step 104, injection into the vitreous humor 38 using a very small bore needle 12 is used to deliver a concentrated solution of nanoparticles 36 in saline to the sites 38 to be treated. In step 106, the eye 30 is then irradiated with a dose of light 34. The wavelength, power, and duration of the light 34 are dependent on the properties of the light-degradable polymer itself. After irradiation, the polymer particle will begin to degrade into small fragments, releasing the therapeutic material encased within the particle until the particle no longer remains intact. Depending on the retention time of the particle in the eye, in step 108 repeat light irradiation doses may be administered for more sustained delivery without further need for invasive injections. For extended treatments, it may be necessary to replenish the available supply of therapeutic. In such a case, in step 110, the process repeats starting with step 104. While such a treatment regimen may involve repeated injections, the frequency of the injections can be reduced with longer intervals between injections compared with existing treatments. The same principles of polymer and thus particle degradation and release can be applied to other administration routes such as topical formulations, contact lens application, or sub-conjunctival injection.

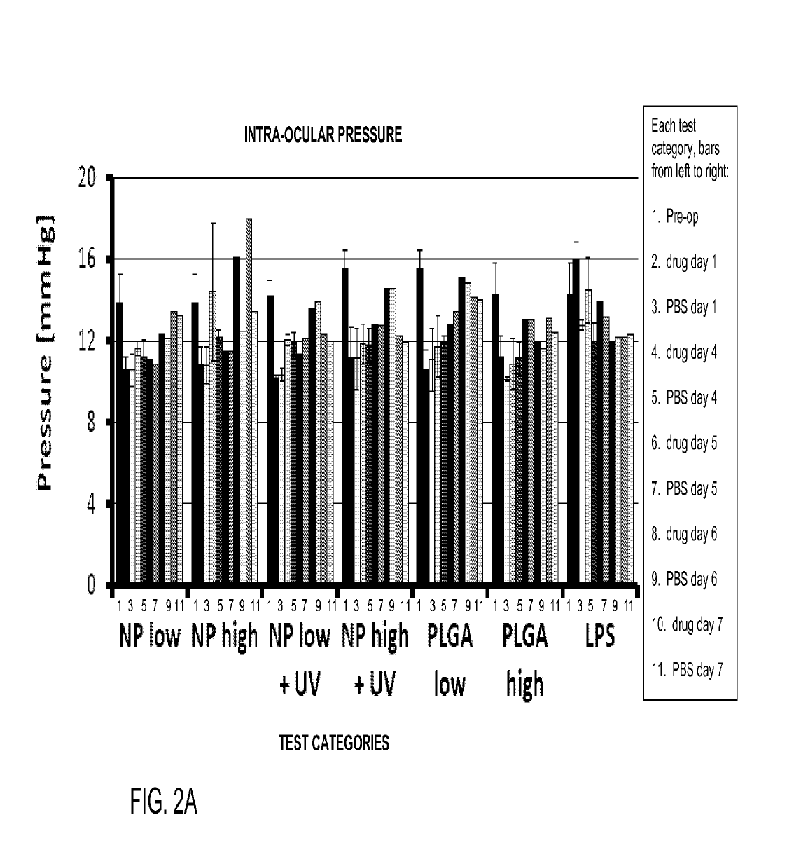

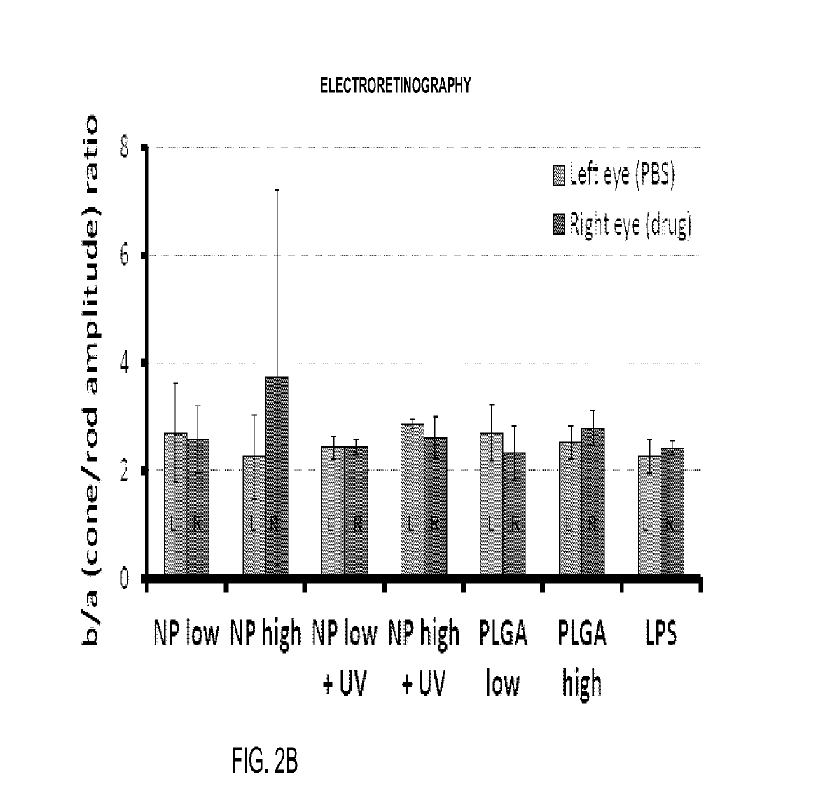

FIG. 1C FIG. 2 is a representative schematic diagram of the components of the light-degradable drug delivery system 10 of the present invention. The light-degradable particles 36 with therapeutic payload (in solution) are injected into the targeted location within eye 30 using syringe 32 with an appropriate small gauge needle. The particles 36 should be sterilize and certified endotoxin-free. The UV light energy 34 from the energy source 14 (a laser, LED, LCD or arc lamp) is directed into delivery device 16 via a delivery channel 18, which may be a fiber optic, articulated arm, or other appropriate optical waveguide. In preferred embodiments, the light source 14 emits UV radiation with a wavelength around 350-365 nm, but may be tunable to allow selection of an appropriate wavelength that is optimized for controlled degradation of the polymer of which the particles are formed. The light source should preferably have adjustable power to modulate the light intensity to avoid damage to the eye. Control system 22 provides a user interface for use by the physician, or assisting nurse or technician, to select the appropriate emission wavelength, intensity, duration and other parameters that may affect the treatment. At the distal end of delivery device 16 is an energy directing means 28 for directing the energy toward the eye 30. The directing means 28 may be one or more optical elements such as a lens or other focusing element, beam shaping optics, slits, apertures, gratings, an array of lenses and other optics or other focusing configuration, which focuses the beam to the depth and area within the eye containing the particles.

In a preferred embodiment, the optical elements may include beam expanding lenses to allow adjustment of the beam spread to cover different size target areas. The invention further includes a kit for delivering therapeutic compounds or materials to the eye in conjunction with an existing light source. The kit includes the light-degradable nanoparticles 36 in solution and syringe 32 for delivering the nanoparticles to the targeted location(s). In an alternative embodiment, where the delivery is made via a contact lens, the kit includes a contact lens impregnated with a solution containing the nanoparticles 36. The contact lens may be made from a material currently in use for drug delivery, such as silicone hydrogel, however, considerations should be made to avoid destabilization of the nanoparticles during polymerization of the lens. In one approach, it may be possible to incorporate the drug-containing nanoparticles into a surfactant that can be loaded into the contact lens, or a nanobarrier may be utilized. For example, a nanobarrier of Vitamin E has been shown to be an effective means for controlling the release of ophthalmic drugs due to its high viscosity. Other methods for incorporating the nanoparticles within a contact lens may be used as long as the ability to irradiate the nanoparticles with the release-triggering light is not impaired, or if some impairment occurs, adjustments should be made to compensate for the impairment. In another embodiment, the nanoparticle solution may be administered as eye drops, where the drug release does not occur until the nanoparticle breakdown is triggered by exposure to light.

The light-degradable polymers can encapsulate a wide range of different possible therapeutics ranging from small molecule drugs to large proteins by tailoring the formulation process.

The particles may be formed using a composition that comprises a multi-photon responsive element covalently linked to a self-immolative backbone subunit. In one embodiment, the multi-photon responsive element is a two-photon responsive element; non-limiting examples of which can be drawn from the bromo-coumarin group. In some embodiments, the composition further comprises a molecular network, and may further comprise a payload. In various embodiments, the molecular network may comprise acrylamide elements and/or PEG elements. In some embodiments the self-immolative backbone subunit is a self-immolative dendrimer oligomer, and/or may comprise an assembled dendritic structure.

One example of an appropriate light-sensitive degradable polymer for forming the nanoparticles is a quinone-methide self-immolative moiety, which can be triggered to degrade through multiple light-sensitive groups along the backbone. Nanoparticles formulated from this polymer are capable of releasing their small molecule payload upon irradiation.

The monomer design is based on the self-immolative quinone-methide system. The cleavage of the triggering group by light induces cyclization of the diamine spacer, which in turn unmasks an unstable quinone-methide moiety. Incorporation of this moiety into a polymer chain such as polymer 2 below causes degradation of the polymer backbone upon irradiation with light.

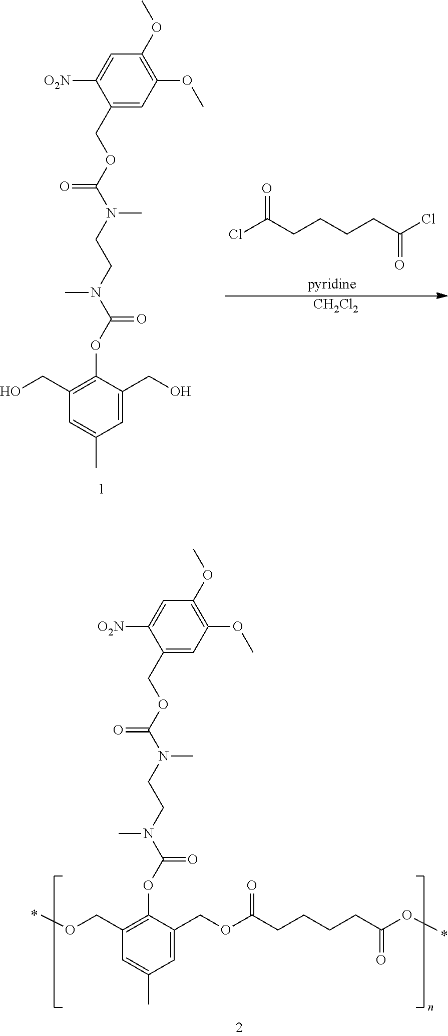

Monomer 1 was synthesized using known techniques. 4,5-Dimethoxy-2-nitrobenzyl alcohol was chosen despite its low two-photon uncaging cross-section (0.01 GM) compared to 4-bromo-coumarins (1 GM) or fluorene-based systems (5 GM).

Monomer 1 was copolymerized with adipoyl chloride to yield a regular copolymer. The low molecular weight oligomers were removed by repeated precipitation of the crude polymer with cold ethanol, yielding the final product with a molecular weight of 65,000 Da and PDI of 1.54 (characterized by GPC relative to polystyrene standards) with 44% yield.

Cleavage of the triggering groups by irradiation at 350 nm and 750 nm, via one and two-photon processes respectively, was monitored by observing the changes in the absorbance spectrum of polymer 2 in acetonitrile/H₂O (9/1). Upon light exposure, the peak at 346 nm, corresponding to 4,5-dimethoxy-2-nitrobenzyl carbamate decreased, while a new peak at 400 nm appeared, corresponding to the cleaved 4,5-dimethoxy-2-nitrosobenzaldehyde. The absorption spectrum remained unchanged after 15 minutes of irradiation with 350 nm light, indicating complete deprotection, while it was necessary to irradiate the system for 5 hours at 750 nm to observe changes in the absorption spectrum, consistent with the low two-photon uncaging cross-section of 4,5-dimethoxy-2-nitrobenzyl group.

The degradation of polymer 2 was studied by GPC and proton NMR in acetonitrile/water solutions. The polymer solutions were exposed to UV light (350 nm) for various periods of time and incubated at 37° C. Samples were removed and analyzed. The degree of polymer degradation showed strong dependence on the irradiation time. The initial drop in molecular weight in the first few minutes after UV irradiation is likely to be mostly due to the loss of the triggering groups, while further reduction in molecular weight is due to the cleavage of the polymer backbone as a result of cyclization and elimination reactions within the self-immolative monomer unit. The difference in the degradation degree is especially evident in the samples irradiated for 5 and 15 minutes: more triggering groups are cleaved. Consequently, the polymer chains degrade into smaller fragments. Although the estimated molecular weights of the fragments level off at 20,000 Da the molecular weight of monomer 1 (m/z=544.19) was estimated by GPC to be 3,500 Da, therefore these fragments may be oligomers. Only a small portion of all the triggering groups needs to be cleaved to induce a reduction in the molecular weight of the polymer.

The cyclization of the diamine linker has been shown to be the rate-determining step of the self-immolation within the quinine-methide unit, and it has been shown to accelerate in the presence of triethylamine. Thus, we measured polymer degradation in the presence of triethylamine and observed an increase in the rate of the polymer degradation. Two-photon irradiation of polymer 2 for 5 hours exhibited a similar degree of degradation as 5 minute one-photon irradiation.

The inventive method includes delivering a payload to ocular tissue, or a selected location within the ocular tissue, which can then be irradiated with an appropriate wavelength of electromagnetic radiation, e.g., light, so as to activate the multi-photon responsive element, which in turn disrupts the composition of the polymer within the selected tissue or in the selected location, thereby releasing the payload. In some embodiments, the radiation used is near infrared light, in other embodiments it may be UV light (˜350 nm).

The light-degradable polymer for use in the inventive method can be formulated so as to amplify its sensitivity to electromagnetic radiation, or light, such as UV light or near infrared light. In some embodiments, a polymer composition for delivery of an ocular therapeutic comprises a multi-photon responsive element and a self-immolative backbone. The composition may be irradiated with electromagnetic radiation, triggering the multi-photon responsive element together with the self-immolative backbone.

A multi-photon responsive moiety may be repetitively embedded in a polymer during or after the synthesis of the polymer. The polymer with the multi-photon responsive element may in turn be used in the formation of materials, nanoparticles, and/or microparticles. When the multi-photon responsive moiety simultaneously absorbs, for example, two photons, changes in the molecular moiety gradually disintegrate the polymer, initiating a domino effect that effectively unravels the entire material, nanoparticle, and/or microparticle. This response is similar to a net in which the cross-linking strands can be selectively removed from a distance, allowing what was trapped within the net to escape through the openings because the surviving strands can no longer, by themselves, retain the former cargo. Thus, incorporation of multi-photon responsive moieties into nanoparticles and/or microparticles during their synthesis establishes sensitivity to multi-photon light stimulation, which, in turn, allows the facile triggering of the fragmentation of the materials, nanoparticles and/or microparticles at selected target sites.

### Example 1

Light-degradable polymers were synthesized as described in International Publication No. WO2011/038117. Initial in vivo testing on a rat model (Sprague-Dawley albino rats) was performed to determine the biocompatibility of the empty polymer particles without encapsulated cargo. This involved intra-vitreous injection of different concentrations of the light-degradable particles (“NP low” and “NP high” test categories in FIG. 2A) as well as different concentrations of nanoparticles composed of a known biocompatible material (PLGA). In each animal, the test material was injected into the right eye while a phosphate buffered solution (PBS) was injected into the left eye as a negative control. Additionally, animals were administered nanoparticle solutions that had previously been irradiated with UV light (˜350 nm) to determine any deleterious effects of the degradation byproducts (“NP low+UV” and “NP high+UV”.) Lipopolysaccharide (LPS) was injected in another group as a positive control as it is known to cause inflammation. Animals were observed for seven days after injection. FIG. 2A illustrates the intra-ocular pressure (IOP) data for these tests, with each group of bars, with the bars proceeding from left to right in the figure, corresponding to pre-operative IOP, drug (nanoparticle) day 1, PBS at day 1 after injection, drug (nanoparticle) day 4 after injection, PBS day 4, drug day 5, PBS day 5 etc. up to day 7 after injection. As indicated, IOP remained at normal levels (10-20 mm Hg) after injection.

Results from this experiment revealed little pervasive negative effects on the health and ocular functions of the animals as a result of the injected nanoparticles. This was determined through three routes: First, visual and microscopic inspection of the (right) eye showed no more reddening or hemorrhaging from the nanoparticle injected eyes compared with PBS buffer (left eye), while LPS caused tearing in the animals over days. Second, the intra-ocular pressure was measured daily. These measurements showed no dips or peaks outside the healthy/normal range for these animals indicating no problems in flow within the eye. Third, electroretinography (ERG) scans were performed on the animals after injection. These results are shown in FIG. 2B. These data indicated little difference between the eyes treated with nanoparticles and those injected only with saline. The ERG data indicate that the photoreceptor cells are healthy and the retina is functioning properly.

In the following examples, small molecules were encapsulated in the light-sensitive polymer (polymer 2 above) and light-triggered release was evaluated both in vitro in different cell lines and in vivo in rat eyes using cell membrane-permeable fluorescent dyes. In initial testing, the particles retained their payload three days post-injection, however, longer-term particle and payload retention may be appropriate in certain situations. To fine-tune the treatment procedure, an irradiance level of 11 mW/cm² of 365 nm UV light was selected to avoid inducing cataract formation in cultured rat lens explants, while still providing enough energy to trigger release from the particles. The light source was an OmniCure® 52000 spot UV curing system device with an appropriate filter. The light source uses a high pressure 200 W mercury vapor short arc lamp and includes filters for selecting light within a range of 320-500 nm range. Other options that may be used for the light source include LEDs, LCDs and lasers.

As will be readily apparent to those in the art, variations in the polymer make-up with dictate which wavelength(s) may be used to degrade the polymer. Thus, while the examples described herein specify UV radiation, visible, near-IR (NIR), IR and other wavelength ranges may be selected depending on the payload to be delivered, the duration and number of iterations of payload delivery.

### Example 2

### In Vitro Results

The dye selected for these studies was Fluorescein Diacetate (FDA), which is an ester of fluorescein. It is cell membrane-permeable and does not fluoresce in its ester form, but once it passes the cell membrane, intracellular esterases cleave the ester bond, releasing fluorescein, a green fluorescing dye. Hence, FDA encapsulated in nanoparticles does not fluoresce, but once it is released and diffuses into the cells, it should be possible to observe green fluorescence. Nanoparticles containing FDA were formulated through an inverse emulsion/solvent evaporation process. The polymer was dissolved in dichloromethane (DCM), and FDA in dimethyl sulfoxide (DMSO), and the two solutions were combined in 7:1 volumetric ratio of DCM to DMSO. The resulting solution was added to a larger volume of 1% solution of polyvinyl alcohol (PVA) and probe sonicated to form the emulsion. The organic solvent was then evaporated under vacuum, and PVA removed by tangential flow ultrafiltration. The resulting solution was then freeze-dried with mannitol as cryoprotectant. The resulting particles were characterized by dynamic light scattering (DLS) and scanning electron microscopy (SEM). The encapsulation efficiency was evaluated by liquid-liquid extraction of FDA from particles and fluorescence measurements. The lyophilized powder was re-suspended either in culture media before use in cells, or saline buffer before use in animal studies. It is worth noting that while the actual drugs may be encapsulated through similar emulsion/solvent evaporation techniques, the actual solvent system will vary depending on the properties of the payload. For hydrophobic payloads, we most often use DCM as the organic phase in the inverse emulsion process, while for hydrophilic payloads we use a double emulsion process to encapsulate the drug.

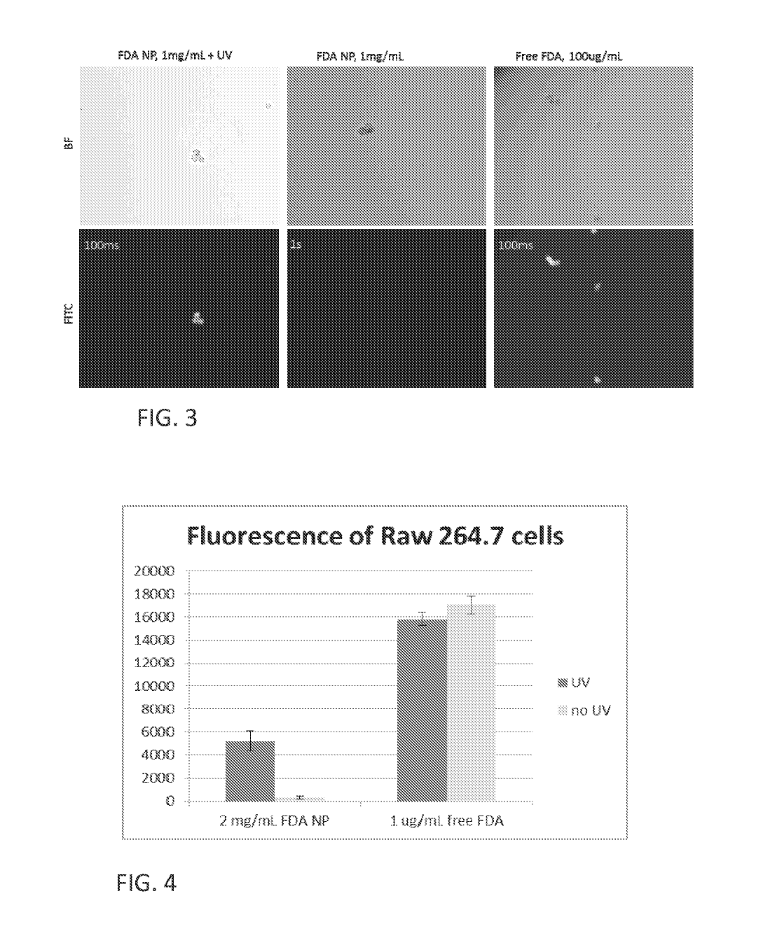

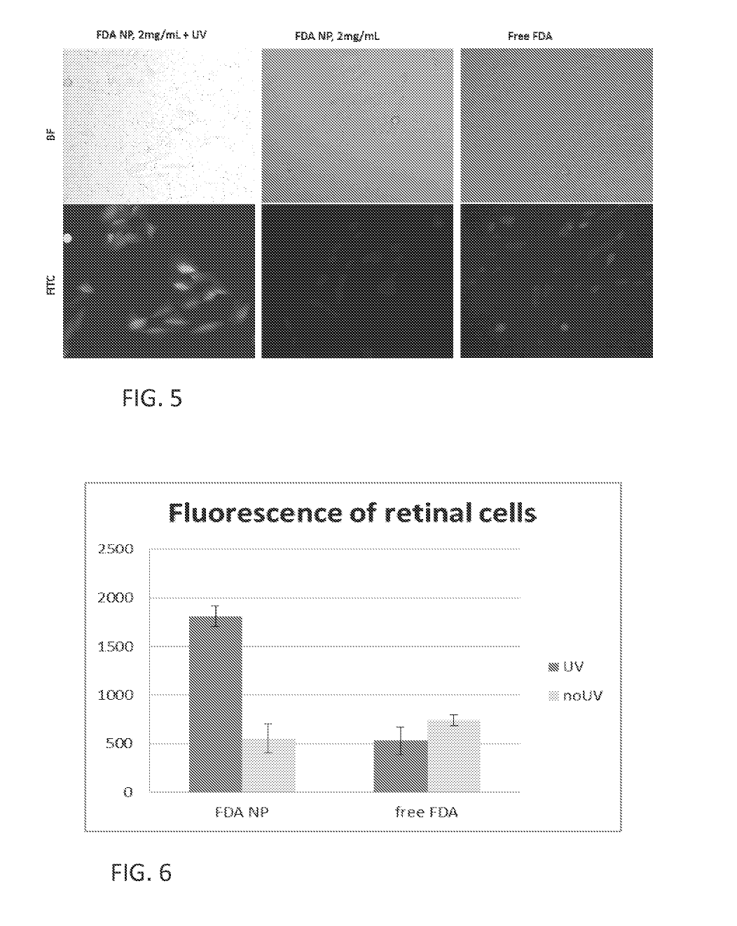

We imaged Raw 264.7 cells incubated with FDA-containing particles with and without irradiation, and incubated with free FDA. As a more quantitative assay, we measured the amount of fluorescence of the cells using a plate reader (same cell count in each well). FIG. 3 is a series of micrographs of the Raw 264.7 macrophage cells incubated with FDA-containing nanoparticles. These images demonstrate triggered release of payload upon irradiation with UV (left) as compared to non-irradiated sample (middle). The right column shows free FDA as a positive control. The top row shows bright field images while the bottom row corresponds to the fluorescein channel. FIG. 5 shows corresponding images for the same test on retinal cells. FIG. 4 is a bar graph showing the measured fluorescence for irradiated and non-irradiated Raw 264.7 cells. The cells in irradiated wells show higher fluorescence due to triggered release of payload (dark bars) as compared to cells in non-irradiated wells (light bars). Irradiation does not cause significant bleaching of FDA. FIG. 6 shows the corresponding results for retinal cells. We found an 18-fold increase in fluorescence upon irradiation as compared to the non-irradiated control in Raw 264.7 macrophage cell line, as well as a 3-fold increase upon irradiation in retinal cell line.

### Example 3

### In Vivo Results

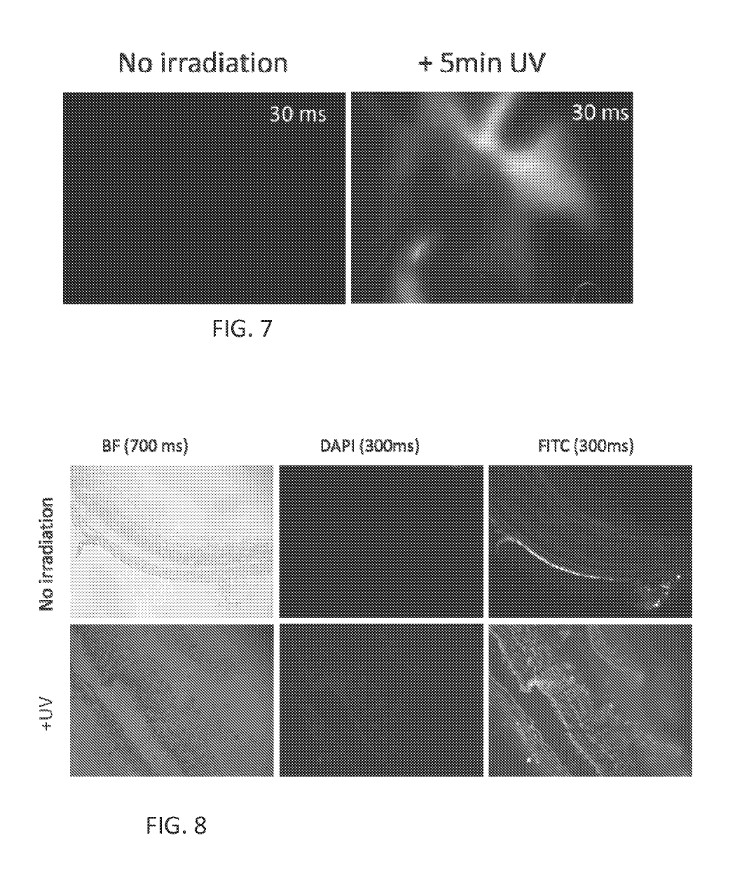

In vivo studies were performed in wild-type Sprague Dawley rats. The nanoparticles were delivered into the vitreous cavity through an intravitreous injection using standard procedures (see, e.g., FIG. 1A). The rat's eyes were irradiated with UV light for five minutes under anesthesia. The animals were sacrificed and their eyes were enucleated 1 hour after irradiation. The cornea was then punctured with a 30 G needle and the eye tissue was fixed by immersing in 4% paraformaldehyde (PFA) for 1 h. The tissue was then either frozen in Optimal Cutting Temperature (OCT) compound to prepare frozen sections, or processed to make retinal flatmounts. We studied the release of the dye in vivo through fluorescence imaging of retinal flat mounts and posterior eye cup frozen sections. As shown in FIG. 7, fluorescent microscope images of flat mounts of retinas from irradiated eyes (right panel) shows significantly higher green fluorescence compared to non-irradiated samples (left panel). The images are merges from FITC (fluorescein isothiocyanate), DAPI (4′,6′-diamidino-2-pheylindole) and Texas Red channels to check for autofluorescence.

FIG. 8 is a series of fluorescent microscope images of cryosections of the posterior ocular cup from UV irradiated and non-irradiated eyes show staining on the inside of the eye. The tissue from the UV irradiated eye (bottom) shows staining in the retina. The non-irradiated eye (upper images) shows some staining outside of the cup but no staining in the retina (upper right). This probably resulted from processing.

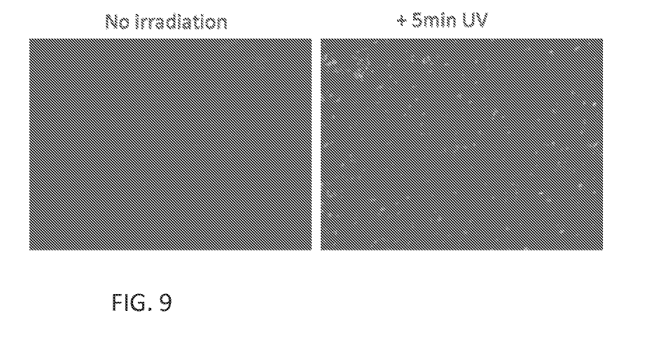

FIG. 9 is microscopic images of a retinal flat mount 3 days post-injection. Retina from the irradiated eye (right) has green fluorescence from FDA, while the non-irradiated control retina (left) shows no staining. DAPI was used as a counter-stain. The images are merged from FITC, DAPI and Texas Red channels.

The present invention provides a system and method for delivery of ophthalmic therapeutics that reduces, or in some cases, may completely eliminate the need for unpleasant and potentially damaging repeated intra-ocular injection. The drug is encapsulated in a light-degradable polymer that can be activated in one or repeated exposures to light having a wavelength that will cause the polymer to breakdown, releasing all or a portion of the drug payload. The invention provides flexibility for patients requiring regular or repeated administration of a therapeutic for an ocular condition, allowing them to perform a portion of the treatment at home, or otherwise outside of the physician's office, by exposing the affected eye to an appropriate light source provided by or prescribed by his or her physician. The nanoparticle-containing solution can be administered by the physician in the office, allowing the patient to activate the drug release as needed or at prescribed intervals using the appropriate light source.

1. A system for delivering a payload to ocular tissue, comprising:
a solution comprising light-degradable nanoparticles encapsulating the payload;
a device configured for introducing the solution into the ocular tissue;
a light source for delivering a beam of light to the ocular tissue;
at least one beam adjusting optical element for controlling focus and beam size within the ocular tissue; and
a system controller for providing control signals to the light source, wherein the control signals comprise selection of an emission wavelength, an emission intensity and an exposure duration, and wherein the emission wavelength is adapted to induce at least particle degradation of the nanoparticles to release the payload to the ocular tissue.

2. The system of claim 1, wherein the device configured for introducing the solution comprises a syringe and needle for intra-ocular injection.

3. The system of claim 1, wherein the device configured for introducing the solution comprises a contact lens having the solution incorporated therein.

4. The system of claim 1, wherein the light source is selected from the group consisting of lasers, LEDs, LCDs and arc lamps emitting in the ultraviolet light range.

5. A method for delivering a payload to ocular tissue comprising the steps of:
synthesizing a particle wherein said particle further comprises a light-degradable polymer and a payload;
incorporating the particle in a solution;
administering the solution to the ocular tissue; and
irradiating the ocular tissue comprising said particle with light having a wavelength adapted to induce degradation of the particle;
wherein the particle is disrupted in situ following absorption of the light.

6. The method of claim 5, wherein the light is ultraviolet light.

7. The method of claim 5, wherein the light is emitted by a light source selected from the group consisting of lasers, LEDs, LCDs and arc lamps emitting in the ultraviolet light range

8. The method of claim 5, wherein the particle comprises a polymer having a self-immolative backbone.

9. The method of claim 5, wherein the step of administering is selected from intra-vitreal injection, sub-conjunctival injection, topical formulation or embedding the particle in a contact lens.

10. A drug-delivery agent for delivering a payload to ocular tissue comprising light-degradable nanoparticles suspended in a solution, wherein the light-degradable nanoparticles are adapted to degrade upon exposure to light having a wavelength adapted to induce degradation in the nanoparticles and release the payload into ocular tissue to which the nanoparticles have been introduced.

11. The drug-delivery agent of claim 10, wherein the nanoparticles are polymers having a self-immolative backbone.

12. A kit for drug delivery to a target area within an eye irradiated by light energy, the kit comprising:
a solution comprising particles formed from a light-degradable polymer, wherein the particles are adapted for encapsulating a therapeutic payload;
a device for administering the solution to a target area within the eye.

13. The kit of claim 12, wherein the device for administering the solution comprises a syringe and needle configured for intra-ocular injection.

14. The kit of claim 12, wherein the device for introducing the solution comprises a contact lens having the solution incorporated therein.

15. The kit of claim 12, wherein the light-degradable polymer is configured to degrade upon exposure to ultraviolet light.

16. The system of claim 1, wherein the at least one beam adjusting optical element is selected from the group consisting of lenses, focusing elements, beam shaping optics, slits, apertures, grating, and combinations thereof.

17. The system of claim 1, wherein the at least one beam adjusting element is configured to control a focal depth of the beam within the ocular tissue.

18. The method of claim 5, wherein the step of irradiating the ocular tissue comprises focusing a beam of light to a depth and area corresponding to a region of ocular tissue to be targeted by the payload.

19. The method of claim 5, wherein the step of incorporating comprises incorporating a plurality of particles in the solution, and wherein the step of irradiating comprises applying the light for a predetermined treatment period selected to disrupt a portion of the plurality of particles, and further comprising, repeating applying the light for one or more predetermined treatment periods selected to disrupt additional particles of the plurality.
